(19) United States
(12) Patent Application Publication
(10) Pub. No.: US 2005/0245772 A1
(43) Pub. Date: Nov. 3, 2005

(54) Derivatives of alcohols and olefins

(76) Inventors: Howard Lam-Ho Fong, Sugar Land, TX (US); Lizbeth Olivia Cisneros Trevino, Tamaulipas (MX); Brendan Dermot Murray, Houston, TX (US); Manuel Luis Cano, Houston, TX (US); Terry Blane Thomason, Houston, TX (US)

Correspondence Address: SHELL OIL COMPANY, P O BOX 2463, HOUSTON, TX, 772522463, US

(21) Appl. No.: 11/116,892

(22) Filed: Apr. 28, 2005

Related U.S. Application Data
(60) Provisional application No. 60/567,038, filed on Apr. 30, 2004.

(51) Int. Cl. C11D 3/37; C07C 27/00

(52) U.S. Cl. 568/893; 510/475

(57) ABSTRACT

Derivatives of branched or linear olefins and alcohols made by halogenation of branched or n-alkanes of the same carbon number, preferably to make primary mono-haloalkanes, which may then be oxidized with metal oxides. The derivatives of primary alcohols of this invention include alcohol ethoxylates, alcohol sulfates, alcohol sulfated ethoxylates, surfactants, including those made with these ethoxylates and/or sulfated ethoxylates, detergents made with these surfactants, oxyalkylated alcohols, oxyalkylated alcohol sulfates, polymethacrylate esters, alkyl amines and their derivatives, linear phthalates, linear adipates, alcohol ether amines, alkyl glycerol ether sulfonates, thioproprionate esters, alkyl polyglucosides, alcohol phosphates, alcohol ether phosphates, esters of fatty acids, alcohol phosphites, and benzophenones. Secondary alcohol derivatives include secondary alcohol ethoxylates, secondary alcohol sulfates, secondary alcohol sulfated ethoxylates, surfactants, including those made with these ethoxylates and/or sulfated ethoxylates, and detergents made with these surfactants. Internal olefins derivatives included within the scope of this invention include alkylbenzene, alkylxylene, detergent alcohols, plasticizer alcohols, alkenyl succinates, ether secondary alcohols, and diols and polyols produced by catalytic dihydroxylation of internal olefins with the use of hydrogen peroxide. Alpha olefin derivatives included within the scope of this invention include internal olefins, alkylbenzene, alkylxylene, alkylphenol, alkylnaphthalene, detergent alcohols, plasticizer alcohols, alkylates of phenylbenzo compounds, alkyl ammonium salts of unsaturated fatty acids, alkyl amines and their derivatives, alpha olefin sulfonates, alkenyl succinates, polyalphaolefins, linear mercaptans, synthetic acids such as based on 1-butene, 1-hexene, or 1-octene, chlorinated alpha olefins, aluminum alkyls, alkyl diphenylether disulfonates, fatty acids, lubricant additives, and ether primary alcohols.

### REFERENCE TO PRIOR APPLICATION

This application claims the benefit of U.S. Provisional application Ser. No. 60/567,038, filed Apr. 30, 2004, the entire disclosure of which is herein incorporated by reference.

### FIELD OF THE INVENTION

This invention relates to alcohols and olefins made by the conversion of linear or branched alkanes and derivatives thereof. More particularly, the invention relates to alcohols and olefins made by a process wherein halogen or a compound containing halogen is used as an intermediate in the conversion of alkanes to alcohols and olefins and derivatives thereof.

### BACKGROUND OF THE INVENTION

Olefins and alcohols can now be produced by a new process which involves the halogenation of paraffins (alkanes) and the oxidation of the mono-haloalkanes produced thereby with metal oxide to produce a mixture of products that contains olefins and alcohols. Such a process, which produces mainly internal olefins and secondary alcohols, is described in U.S. Pat. Nos. 6,462,243, 6,465,699, 6,472,572, 6,486,368, and 6,465,696. Suitable processes for making alpha olefins and primary alcohols by the halogenation of alkanes) and the oxidation of the primary mono-haloalkanes produced thereby with metal oxide are also described in copending, commonly assigned patent applications entitled PROCESS TO CONVERT LINEAR ALKANES INTO ALPHA OLEFINS and PROCESS TO CONVERT ALKANES INTO PRIMARY ALCOHOLS, Ser. No. ______ and Ser. No. ______, both filed Apr. ______, 2005.

### SUMMARY OF THE INVENTION

This invention relates to branched or linear alpha olefins (AO) and alcohols made by halogenation of branched or n-alkanes of the same carbon number, preferably to make primary mono-haloalkanes which are then oxidized with metal oxides, and derivatives thereof. Carbon numbers of particular interest are C₄-C₂₂, preferably C₆ to C₁₈, more preferably C₆ to C₁₄, and most preferably C₆ to C₁₀.

The derivatives of primary alcohols of this invention include alcohol ethoxylates, alcohol sulfates, alcohol sulfated ethoxylates, surfactants, including those made with these ethoxylates and/or sulfated ethoxylates, detergents made with these surfactants, oxyalkylated alcohols, oxyalkylated alcohol sulfates, polymethacrylate esters, alkyl amines and their derivatives, linear phthalates, linear adipates, alcohol ether amines such as C₈-C₁₀, alkyl glycerol ether sulfonates, thioproprionate esters, alkyl polyglucosides, alcohol phosphates, alcohol ether phosphates, esters of fatty acids, alcohol phosphites, and benzophenones.

Secondary alcohol derivatives include secondary alcohol ethoxylates, secondary alcohol sulfates, secondary alcohol sulfated ethoxylates, surfactants, including those made with these ethoxylates and/or sulfated ethoxylates, and detergents made with these surfactants.

Internal olefin derivatives included within the scope of this invention include alkylbenzene, alkylxylene, detergent alcohols, plasticizer alcohols, alkenyl succinates, ether secondary alcohols, and diols and polyols produced by catalytic dihydroxylation of internal olefins with the use of hydrogen peroxide.

Alpha olefin derivatives included within the scope of this invention include internal olefins, alkylbenzene, alkylphenol, alkylnaphthalene, detergent alcohols, plasticizer alcohols, alkylates of phenylbenzo compounds, alkyl ammonium salts of unsaturated fatty acids, alkyl amines and their derivatives, alpha olefin sulfonates, alkenyl succinates, polyalphaolefins, linear mercaptans, synthetic acids such as based on 1-butene, 1-hexene, or 1-octene, chlorinated alpha olefins, aluminum alkyls, alkyl diphenylether disulfonates, fatty acids such as C₇-C₉, lubricant additives, and ether primary alcohols.

The olefins and alcohols for derivatization are made by a process which includes the steps of a) halogenating linear alkanes, branched alkanes, or a mixture of linear and branched alkanes to produce a mixture of mono-haloalkanes, preferably primary mono-haloalkanes (i.e., alkanes with one halogen attached in the primary position), and hydrogen halide; b) separating the hydrogen halide from the mixture of step a) and optionally neutralizing it with a metal oxide to produce a partially halogenated metal oxide and/or metal halide which may be regenerated to halogen and metal oxide; c) separating the mono-haloalkanes, preferably primary mono-haloalkanes, from the mixture of step a); d) reacting the separated mono-haloalkane, preferably primary mono-haloalkane, with a metal oxide (and water if an alcohol is being produced) to convert the mono-haloalkane, preferably primary mono-haloalkane, to a mixture of products that contains olefins, alcohols, and unconverted mono-haloalkanes, preferably primary mono-haloalkane, and a partially halogenated metal oxide and/or metal halide which optionally may be regenerated to halogen (such as Br₂) and/or acid (such as HCl) and a metal oxide by reaction with air, oxygen or gas mixtures containing oxygen gas; and e) removing the unreacted mono-haloalkane, preferably primary mono-haloalkane, from the reaction mixture and then purifying the olefins and/or alcohols.

### DETAILED DESCRIPTION OF THE INVENTION

The olefins and alcohols described herein can be used themselves in a wide variety of applications. These applications are also within the scope of this invention. For example, the following uses and applications are applicable:

Internal olefins can be used as drilling fluids for oilfield drilling muds (as described in U.S. Pat. No. 5,589,442 which is herein incorporated by reference)

Alpha olefins can be used as co-monomers for polyethylene production (linear low density polyethylene (LLDPE), high density polyethylene (HDPE) and in drilling fluids for oilfield drilling muds (as described in U.S. Pat. No. 5,432,152 which is herein incorporated by reference)

Secondary alcohols can be used as solvents, emollients, and conditioners in cosmetic and toiletry applications; components in rolling oil formulations used in the rolling of metal foils or sheet stock such as aluminum foil; emulsifying aids in the preparation and stabilization of pharmaceutical dispersions and emulsions; micelle control agents in the production of plastics with emulsion polymerization; and defoaming agents (e.g., in the paper industry).

Primary alcohols can be used as solvents, emollients, and conditioners in cosmetic and toiletry applications; components in rolling oil formulations used in the rolling of metal foils or sheet stock such as aluminum foil; emulsifying aids in the preparation and stabilization of pharmaceutical dispersions and emulsions; micelle control agents in the production of plastics with emulsion polymerization; and defoaming agents (e.g., in the paper industry).

Compositions within the scope of this invention include a wide variety of derivatives of the internal and alpha olefins made by the halogenation process described herein. Other compositions within the scope of this invention include a wide variety of derivatives of the primary and secondary alcohols made by the halogenation process described herein.

### Alcohols

The compositions of the present invention include alcohol ethoxylates which are derived from the alcohols made by the above process which are then ethoxylated by adding ethylene oxide to a mixture of the alcohols and an acid catalyst. The temperature for this reaction may preferably be in the range of about 20° C. to about 160° C. and may preferably be carried out at atmospheric or higher pressure. The acidic catalysts may include, in a broad sense, the substances classified in the art as Lewis acids or Friedel-Crafts catalysts. Specific examples of these catalysts are the halides, boron, antimony, tungsten, aluminum, iron, nickel, tin, zinc, titanium, and molybdenum. Complexes of such halides with, for example, alcohols, ethers, carboxylic acids, and amines, have also been reported as effective acidic ethoxylation catalysts. Still other representative examples are sulfuric and phosphoric acids and the perchlorates of magnesium, calcium, manganese, nickel, and zinc. Other possible catalysts include metal oxylates, sulfates, phosphates, carboxylates, and acetates, of the alkali metal fluoroborates, of zinc titanate, and of the zinc salt of benzene sulfonic acid. Preferably, but necessarily, the amount of acid catalysts is on the order of about 0.01 to about 5.0 percent by weight, based on the alcohol reactant. Methods for making alcohol ethoxylates from alcohols are described in U.S. Pat. Nos. 6,150,322 and 5,849,960 and US Published Patent Application No. 2002/0183567, which are herein incorporated by reference. Sulfates of these ethoxylated alcohols are also within the scope of this invention. Similar sulfates can also be derived directly from the olefins made by the process above. These sulfates may be made by reacting the olefin or alcohol with sulfuric acid and then neutralizing the reaction product with an appropriate base. For example, sulfuric acid of about 80 to about 98 percent concentration is mixed with an olefin at a temperature in the range from about 3° C. to about 15° C. The sulfation produces a mixture of alkyl sulfuric acids wherein the majority of the molecules have sulfuric acid ester linkages to the alkyl group at a terminal carbon atom. Methods for making alcohol ethoxysulfates are described in U.S. Pat. Nos. 6,150,322 and 5,849,960 and US Published Patent Application No. 2002/0183567, which are herein incorporated by reference. These references also describe how to make alcohol sulfates within the scope of this invention.

Other alcohol derivatives which are within the scope of this invention include:

Secondary alcohols can be used to make:

Secondary alcohol sulfates

Secondary alcohol ethyoxylates

Secondary alcohol ethoxysulfates

Primary alcohols can be used to make:

Alcohol sulfates as described above
Uses: laundry detergents, dishwashing liquids, other household cleaners, personal care products such as shampoos, bubble baths, and toilet soaps, institutional and commercial cleaning products, emulsifier for herbicide applications, emulsifiers for polymerization

Alcohol ethoxylates as described above
Uses: laundry detergents, industrial, institutional, and commercial cleaning products, textile applications, emulsifiers for herbicide applications, emulsifiers for polymerization

Alcohol ethoxysulfates as described above
Uses: laundry detergents, dishwashing liquids, other household cleaners, personal care products such as shampoos, bubble baths, and toilet soaps, institutional and commercial cleaning products, emulsifier for herbicide applications, emulsifiers for polymerization

Oxyalkylated alcohols (e.g., propylene oxide, butene oxide, or ethylene oxide) (as described in U.S. Pat. Nos. 6,150,322 and 5,849,960 and US Patent Application 2002/0183567, which are herein incorporated by reference)
Uses: Demulsifiers, antifoaming agents for oilfield and refinery applications

Oxyalkylated alcohol sulfates (e.g., propylene oxide, butene oxide, or ethylene oxide) (as described in U.S. Pat. Nos. 6,150,322 and 5,849,960 and US Patent Application 2002/0183567, which are herein incorporated by reference)
Uses: Demulsifiers, antifoaming agents for oilfield and refinery applications

Polymethacrylate esters
Uses: automotive and aircraft lubricating oils (mainly in transmission and hydraulic fluids; function as viscosity index improvers, pour-point depressers, and polymeric dispersants)

Alkyl amines (and derivatives)
Uses: Converted to alkylbenzyldimethylammonium chlorides (active ingredients in fungicides, algaecides, biocides, sanitizers, disinfectants, hair conditioners and shampoos), fatty amine oxides (surfactants for light-duty dishwashing liquids, household cleaners, and personal care products), and alkylbetaines

Linear phthalates
Uses: Calendering (coated fabrics and sheet goods), typical applications include swimming pool liners, roofing membranes, automotive body side molding, tarpaulins and wire and cable jacketing

Linear adipates
Uses: Typically blended with general-purpose phthalates to improve low temperature properties of polyvinylchloride and vinyl chloride/vinyl-acetate copolymer emulsions, especially for dispersions, film and extrusions, applications include automotive accessories, gaskets, flexible hoses and tubing

Alcohol ether amines (C8-C10)
Uses: Mining industry

Alkyl glyceryl ether sulfonates
Uses: Foam-boosting surfactant for light-duty dishwashing, shampoos, and combination soap-synthetic toilet bars

Thiodiproprionate esters
Uses: Antioxidants (particularly effective in stabilizing polyolefins), oxidation inhibitors in elastomers and cellulose acetate

Alkyl polyglucosides
Uses: Nonionic surfactant for laundry, hand-dishwashing, and personal care products

Alcohol phosphates
Uses: Textile applications as lubricants or wetting agents

Alcohol ether phosphates
Uses: Textile applications as lubricants or wetting agents

Esters of fatty acids
Uses: Emollients for use in cosmetic and pharmaceutical applications

Alcohol phosphites
Uses: Antioxidants and stabilizers in plastics

Benzophenones
Uses: Ultraviolet light absorbers or stabilizers for polyolefins and polyvinylchloride

### Olefins

Derivatives of internal and alpha olefin made by the halogenation process described herein are also within the scope of this invention.

Internal olefins can be used to make:

Alkylbenzene, which can be converted to alkylbenzene sulfonate

Alkylxylene
Use: enhanced oil recovery

Detergent alcohols (as described in U.S. Pat. Nos. 6,150,322 and 5,849,960 and US Patent Application 2002/0183567, which are herein incorporated by reference)

Plasticizer alcohols (C6-C11) (as described in U.S. Pat. Nos. 6,150,322 and 5,849,960 and US Patent Application 2002/0183567, which are herein incorporated by reference)

Alkenyl succinates (alkyl succinic anhydrides)
Uses: Paper sizing, lube oil additives, detergents, leather treatment, food, curing agents for epoxy resins, corrosion inhibitors in non-aqueous lubrication oils, and intermediates in the preparation of alkyd or unsaturated resins

Ether secondary alcohols (as described in U.S. Pat. No. 6,706,931 B2, which is herein incorporated by reference)

Diols and polyols can be produced by catalytic dihydroxylation of internal olefins with the use of hydrogen peroxide.
A recent publication (Angew. Chem. International Edition, 2003, 42, pages 5623-5625) teaches that a wide range of olefins can be converted to diols. For example, 1-hexene is converted to 1,2 hexanediol. 2-hexene is converted to 2,3-hexanediol. Branching is not a problem as 2-methyl-2-pentene is converted 2-methyl-2,3-pentanediol and 2,3-dimethyl-2-butene is converted 2,3-dimethyl-2,3-butanediol. Dienes such as 1,7-octadiene can be converted into 1,2,7,8-octanetetraol.
The reactions could be performed using olefins (or di-olefins if made) via the methods described in U.S. Pat. Nos. 6,462,243, 6,465,699, 6,472,572, 6,486,368, and 6,465,696 and in copending, commonly assigned patent applications entitled PROCESS TO CONVERT LINEAR ALKANES INTO ALPHA OLEFINS and PROCESS TO CONVERT ALKANES INTO PRIMARY ALCOHOLS, Ser. No. ______ and Ser. No. ______, both filed Apr. ______, 2005, all of which are herein incorporated by reference.
Thus the olefins made via the halogenation/metathesis route can serve as precursors to diols (and higher hydroxylated species). These diols can be used in a wide range of commercial products or as intermediates to other chemicals.
   Alpha olefins can be used to make:

Internal olefins
Uses: Drilling fluids, feedstock to manufacture alkylbenzene, feedstock to manufacture primary or secondary alcohols, feedstock to manufacture akenylsuccinic anhydrides (ASA)

Alkylbenzene, which can be converted to alkylbenzene sulfonate (LAS)
Uses: LAS is used as a surfactant in household and industrial detergent formulations, lubricants

Other alkyl aromatics including alkylxylene, alkylphenol, and alkylnaphthalene

Detergent alcohols (as described in U.S. Pat. Nos. 6,150,322 and 5,849,960 and US Patent Application 2002/0183567, which are herein incorporated by reference)
Uses: Derivatized to make alcohol sulfates, alcohol ethoxylates, and alcohol ethoxysulfates; uses for these products are listed above

Plasticizer alcohols (C6-C11) (as described in U.S. Pat. Nos. 6,150,322 and 5,849,960 and US Patent Application 2002/0183567, which are herein incorporated by reference)

Alkylates of phenylbenzo compounds
Uses: UV absorbers

Alkyl ammonium salts of unsaturated fatty acids
Uses: Wetting agents, dispersion additives

Alkyl amines (and derivatives)
Uses: Converted to alkylbenzyldimethylammonium chlorides (active ingredients in fungicides, algaecides, biocides, sanitizers, wood preservatives, and disinfectants; and hair conditioners and shampoos), fatty amine oxides (surfactants for light-duty dishwashing liquids, household cleaners, and personal care products), and alkylbetaines

Alpha olefin sulfonates
Uses: Anionic surfactant for laundry powders, personal care (liquid hand soaps and shampoos), industrial applications (emulsion polymerization, oil field applications, liquid hand dishwashing detergents)

Alkenyl succinates (alkyl succinic anhydrides)
Uses: Paper sizing, lube oil additives, detergents, leather treatment, food, curing agents for epoxy resins, corrosion inhibitors in non-aqueous lubrication oils, and intermediates in the preparation of alkyd or unsaturated resins

Polyalphaolefins
Uses: Base stock material for synthetic lubricants for automotive, industrial, and aviation applications; and drag reducing compounds for flow improvement in pipes

Linear mercaptans
Uses: Agricultural chemicals intermediate, polymer modification

Synthetic acids (based on 1-butene, 1-hexene, 1-octene)

Chlorinated alpha olefins
Uses: Coolants and lubricants for the metalworking industry, secondary plasticizers for polyvinylchloride, flame retardants

Aluminum alkyls
Uses: Feedstock for the production of Ziegler-Natta catalysts used for olefin polymerization, production of organotins, stabilizers for polyvinylchloride, biocides

Alkyl dephenylether disulfonates
Uses: Emulsion polymerization, acid dye leveler, bleach solutions, cosurfactant in machine dishwashing liquids and gels, crystal growth modifiers, surfactant in agricultural chemicals

Fatty acids (C7-C9)

Lubricant additives [synthetic heavy alkylate (LAS), phenates (alkylated phenols), sulfurized linear alpha olefins, alkylnaphthalenes]

Ether primary alcohols (as described in U.S. Pat. No. 6,706,931 B2, which is herein incorporated by reference)

Diols and polyols can be produced by catalytic dihydroxylation of alpha olefins with the use of hydrogen peroxide.
A recent publication (Angew. Chem. International Edition, 2003, 42, pages 5623-5625) teaches that a wide range of olefins can be converted to diols. For example, 1-hexene is converted to 1,2 hexanediol. 2-hexene is converted to 2,3-hexanediol. Branching is not a problem as 2-methyl-2-pentene is converted 2-methyl-2,3-pentanediol and 2,3-dimethyl-2-butene is converted 2,3-dimethyl-2,3-butanediol. Dienes such as 1,7-octadiene can be converted into 1,2,7,8-octanetetraol.
The reactions could be performed using olefins (or di-olefins if made) via the methods described in U.S. Pat. Nos. 6,462,243, 6,465,699, 6,472,572, 6,486,368, and 6,465,696 and in copending, commonly assigned patent applications entitled PROCESS TO CONVERT LINEAR ALKANES INTO ALPHA OLEFINS and PROCESS TO CONVERT ALKANES INTO PRIMARY ALCOHOLS, Ser. No. ______ and Ser. No. ______, both filed Apr. ______, 2005, all of which are herein incorporated by reference.
Thus the olefins made via the halogenation/metathesis route can serve as precursors to diols (and higher hydroxylated species). These diols can be used in a wide range of commercial products or as intermediates to other chemicals.

Anionic and nonionic surfactants are also within the scope of the present invention. These surfactants can be made from the olefins and alcohols made by the process described herein and can be used as follows:

Surfactants made from primary alcohols can be used in:

Detergents for laundry—liquid or granulated (as described in U.S. Pat. Nos. 6,150,322 and 5,849,960 and US Patent Application 2002/0183567, which are herein incorporated by reference)

Detergent for liquid dishwashing

Detergent for liquid soaps, shampoos, or scouring agents

Surfactants made from secondary alcohols can be used in:

Detergents for laundry—liquid or granulated (as described in U.S. Pat. Nos. 6,150,322 and 5,849,960 and US Patent Application 2002/0183567, which are herein incorporated by reference)

Detergent for liquid dishwashing

Detergent for liquid soaps, shampoos, or scouring agents

Surfactants made from internal olefins can be used in:

Detergents for laundry—liquid or granulated (as described in U.S. Pat. Nos. 6,150,322 and 5,849,960 and US Patent Application 2002/0183567, which are herein incorporated by reference)

Detergent for liquid dishwashing

Detergent for liquid soaps, shampoos, or scouring agents

Surfactants made from alpha olefins can be used in:

Detergents for laundry—liquid or granulated (as described in U.S. Pat. Nos. 6,150,322 and 5,849,960 and US Patent Application 2002/0183567, which are herein incorporated by reference)

Detergent for liquid dishwashing

Detergent for liquid soaps, shampoos, or scouring agents

The olefins and alcohols to be derivatized may be made by a process to convert alkanes directly to these valuable products. Linear alkanes, branched alkanes, or a combination of linear and branched alkanes are converted via halogenation to a mixture of primary mono-haloalkanes, internal mono-haloalkanes, unreacted alkanes, hydrogen halide, and possibly multi-haloalkanes. Halogenation can be carried out thermally or catalytically (for example in a conventional reactor, in a catalytic distillation (CD) column, etc.), and with or without the use of a support intended to promote shape selectivity.

Halogenation processes that produce mono-haloalkanes for use primarily in the production of internal olefins and secondary alcohols are described in U.S. Pat. Nos. 6,462,243, 6,465,699, 6,472,572, 6,486,368, and 6,465,696, which are herein incorporated by reference. A bromine-containing compound is used to convert alkanes to alcohols or olefins by reaction with oxygen. An alkane reacts with bromine to form a mono-haloalkane which is then reacted with a metal oxide to form an alcohol or olefin and metal bromide. The leftover metal oxide and the metal bromide are regenerated by reaction with oxygen.

Halogenation processes that preferentially produce primary mono-haloalkanes (e.g., catalytic halogenation at lower temperatures, thermal halogenation at higher temperatures, etc.) are preferred. Preferred halogens are chlorine, bromine, and iodine. Particularly preferred is chlorine. Particularly preferred processes that produce primary mono-haloalkanes and thus alpha olefins and primary alcohols are described in copending, commonly assigned patent applications entitled PROCESS TO CONVERT LINEAR ALKANES INTO ALPHA OLEFINS and PROCESS TO CONVERT ALKANES INTO PRIMARY ALCOHOLS, Ser. No. ______ and Ser. No. ______, both filed Apr. ______, 2005, which are herein incorporated by reference. These processes are described in more detail below.

Thermal halogenation may be carried out by introducing the halogen and the alkane to a reactor and heating the reactants within a temperature range for thermal halogenation of from about 100° C. to about 400° C. As stated above, catalytic halogenation may be carried out at lower temperature, such as from about 25° C. to about 400° C. Catalysts which can be used include compounds and/or complexes containing Ti, Zr, Hf, V, Nb, Ta, Cr, Mo, W, Mn, Re, Fe, Ru, Co, Rh, Ir, Ni, Pd, Pt, Cu, Ag, Au, Zn, Cd, B Al, Ga, In, Tl, Si, Ge, Sn, Pb, P, Sb, Bi, S, Cl, Br, F, Sc, Y, Mg, Ca, Sr, Ba, Na, Li, K, O, La, Ce, Pr, Nd, Sm, Eu, Gd, Tb, Er, Yb, Lu and Cs or mixtures thereof. The amount of catalyst used will vary with the specific catalyst used and the reaction conditions selected but will range from about 0.00001 grams to about 100 grams of catalyst per gram of alkane passed over the catalyst per hour. The reaction may also be conducted in the presence of a diluent such as nitrogen, helium or argon. The process can be conducted at pressures ranging from about 0.1 atm to about 100 atm pressure.

To improve the selectivity to primary mono-halogenated products, the halogenation step may be conducted in the presence of a shape selective catalyst that favors halogenation at the ends of the alkane more than at internal positions of the alkane. The selective bromination of halo-aromatic compounds is known to occur with some zeolite catalysts, such as described by Th. M. Wortel et al. in the Journal of Catalysis 60,110-120 (1979), which is herein incorporated by reference. The ability to improve the selectivity to primary mono-halogenated alkanes is desirable. By selecting a molecular sieve with a pore diameter close to that of the critical diffusion diameter of the alkane to be halogenated in this process, the selectivity to primary mono-halogenated alkane can be increased.

The mixture of primary mono-haloalkanes, other mono- and multi-haloalkanes, unreacted alkanes, and hydrogen halide may preferably be transferred to a separation train that isolates the primary mono-haloalkanes from the mixture. The separation train may include (1) a distillation or other appropriate separation step to recover hydrogen halide, (2) a distillation or other appropriate separation step (or multiple steps) to separate unreacted alkanes, multi-haloalkanes, and mono-haloalkanes, and (3) an additional separation step to separate primary mono-haloalkanes from internal mono-haloalkanes. It may be desirable to isomerize the internal mono-haloalkanes to primary mono-haloalkanes or to convert the mono and multi-haloalkanes to olefins or alcohols via dehydrohalogenation or back to alkanes via dehalogenation/hydrogenation. The alkanes converted in this manner can be recycled back to the primary halogenation reactor and/or a disproportionation reactor.

The unreacted alkanes may also be recycled to the primary halogenation reactor and/or a disproportionation reactor. The multi-haloalkanes may be recycled to the primary halogenation reactor or may be recycled to a separate disproportionation reactor. The disproportionation reactor converts some of the multi-haloalkanes in the presence of alkanes to mono-haloalkanes. When the multi-haloalkanes are recycled to the halogenation reactor and fresh alkanes are also sent to the reactor, the halogenation reactor also serves as a disproportionation reactor. If a separate disproportionation reactor is used, the resulting reaction mixture of multi-haloalkanes and mono-haloalkanes is then recycled to the separation train. The internal mono-haloalkanes may be recycled to the primary halogenation reactor or may be recycled to an isomerization reactor to convert some of the internal mono-haloalkanes to primary mono-haloalkanes. If an isomerization reactor is used, the resulting reaction mixture of internal mono-haloalkanes and primary mono-haloalkanes is then recycled to the separation train.

Suitable separation schemes include distillation, extractive distillation, adsorption, melt crystallization, and others. For the primary and internal mono-haloalkanes, separation, distillation, and melt crystallization are particularly preferred. The ability to separate the primary mono-haloalkanes from the internal mono-haloalkanes may also be facilitated by the formation of adducts. For some carbon chain lengths (C₆-C₁₀), distillation is preferred because of differences in boiling points (and as a result, relative volatilities). For other carbon chain lengths (C₁₁-C₁₆), melt crystallization is preferred because of the substantial freezing point difference between primary and internal mono-haloalkanes.

Distillation can be used to separate many of the products of the halogenation reaction because alkanes, mono-haloalkanes, and multi-haloalkanes of the same carbon number boil at temperatures that are quite different. For example, at 760 torr n-hexane boils at 69° C., mono-chlorohexanes boil at between 122-135° C., and di-chlorohexanes boil around 160-205° C. The bromoalkanes boil at even higher temperatures, mono-bromohexanes boil at between 141-155° C., and di-bromochlorohexanes boil around 180-245° C. Hexenes boil at temperatures below that of mono-halohexanes and di-halohexanes. Similar trends are seen with other carbon numbers. For example, at 760 torr n-octane boils at 126° C., mono-chlorooctanes boil around 165-185° C., and di-bromooctanes boil above 225° C. Branched halogenated alkanes can also be separated from non-halogenated branched alkanes by distillation. For example, 3-chloro-methylheptane boils at 174° C. while 3-methylheptane boils at 115-118° C. at a pressure of 760 torr.

The hydrogen-halide produced in the halogenation reactor may be separated and neutralized with a metal oxide or mixture of metal oxides to produce a partially or fully halogenated metal oxide and/or metal halide or mixture of partially or fully halogenated metal oxides and/or metal halides which may then be converted to halogen and metal oxide (or mixture of metal oxides) for recycle using air, oxygen or gas mixtures containing oxygen gas. These mixtures may include blends of oxygen with nitrogen, argon or helium.

Engineering configurations to carry out this hydrogen halide neutralization process include a single reactor, parallel reactors, two reactors (one to trap hydrogen halide and one to regenerate metal-halide), among others. Multiple reactors in series may also be utilized. A number of materials are known to trap or neutralize acidic hydrogen halides. These include bases such as alkali and alkaline earth hydroxides or mixtures thereof. For example, KataLeuna Lime, a mixture of calcium hydroxide and sodium hydroxide can purify hydrocarbon streams containing HCl.

Metal oxides or partially halogenated metal oxides which may be used in this step and in the metathesis reaction below, include oxides or oxyhalides of the following metals: Ti, Zr, Hf, V, Nb, Ta, Cr, Mo, W, Mn, Re, Fe, Ru, Co, Rh, Ir, Ni, Pd, Pt, Cu, Ag, Au, Zn, Cd, B, Al, Ga, In, Tl, Si, Ge, Sn, Pb, P, Sb, Bi, S, Cl, Br, F, Sc, Y, Mg, Ca, Sr, Ba, Na, Li, K, La, Ce, Pr, Nd, Sm, Eu, Gd, Tb, Er, Yb, Lu, and Cs or mixtures thereof. The amount of catalyst used will vary with the specific catalyst used and the reaction conditions selected but may range from about 0.00001 grams to about 100 grams of catalyst per gram of alkane passed over the catalyst per hour. The reaction may also be conducted in the presence of a diluent such as nitrogen, helium and argon. The process may be conducted at pressures ranging from about 0.1 atm to about 100 atm pressure.

The primary mono-haloalkane that is isolated in the separation train may be reacted (oxidized) in a metathesis (oxidation) reactor with a selected metal oxide or a combination of metal oxides to convert the primary mono-haloalkane to a mixture of products that contains alpha olefins, alcohols, the unconverted primary mono-haloalkanes, and possible other reaction products. The metal oxide or combination of metal oxides may be selected in order to maximize the amount of alpha olefin and/or alcohol produced. Reaction conditions such as residence time, temperature, and reaction phase (such as solid-gas or solid-liquid) may be selected to maximize alpha olefin and/or alcohol production. The same metal oxide or combination of metal oxides may be able to produce preferentially different products (such as ethers and olefin oxides instead of alcohols or olefins) depending on the reaction conditions. For example, shorter residence time, lower temperatures, and solid-gas phase reaction tend to preferentially produce olefins over alcohols.

The metal oxide or metal oxides used in the metathesis reactor may or may not be different from the one(s) used in the neutralization of the hydrogen halide. The metal oxide is normally partially converted (but it could be totally converted if desired) to a metal halide and or partially halogenated metal oxide. The unreacted primary mono-haloalkane may be removed from the alpha olefin or alcohol and possible other reaction products. Recovered alpha olefin or alcohol is further purified, as needed, to obtain the desired final product. A purification train may be used to isolate the alpha olefin or alcohol product. Suitable purification schemes may include distillation, adsorption, melt crystallization, and others. The unconverted primary mono-haloalkane may preferably be recycled to the metathesis reactor.

The metal halide and/or partially halogenated metal oxide may be regenerated to a metal oxide or a mixture of metal oxides and halogen (e.g. Cl₂) by using air, oxygen, or gas mixtures containing oxygen gas. These mixtures may include blends of oxygen with nitrogen, argon or helium. The liberated halogen (e.g. Cl₂) may be recycled to the halogenation reactor. If water is also present in the system, hydrogen halide may also be produced. The regeneration of the metal halide and/or partially halogenated metal oxide to metal oxide and halogen may be accomplished with various reactor configurations including a separate regeneration reactor, in situ with a combined regeneration/metathesis reactor wherein the regeneration gas (such as air oxygen, etc) flow and primary alkane feed flow are alternated, in situ regeneration with a multiple metathesis reactor configuration in a fixed bed mode, etc. The same steps may be used when a combination of metal oxides and/or partially halogenated metal oxides are used.

The remaining mixture of primary mono-haloalkanes, internal mono- and multi-haloalkanes, and unreacted alkanes may be transferred to a separation step (or steps) in which the unreacted alkane and the multi-haloalkanes may be removed and then recycled to the halogenation reactor. The remaining mixture of mono-haloalkanes (primary and internal) may be transferred to a separation step to isolate the primary mono-haloalkanes. The internal mono-haloalkanes may be recycled to the halogenation reactor.

The primary mono-haloalkane may be reacted in a metathesis reactor with a selected metal oxide or a combination of metal oxides to convert the primary mono-haloalkane to a mixture of products that contains alpha olefins, alcohols, and unconverted primary mono-haloalkanes. The metal oxide(s) may be partially converted to a metal halide(s) and/or partially halogenated metal oxide(s). A purification train may be used to separate alpha olefins and/or alcohols from unconverted primary mono-haloalkanes and other reaction products. The unconverted primary mono-haloalkane may be recycled to the metathesis reactor. The metal halide(s) and/or partially halogenated metal oxide(s) may be regenerated to metal oxide(s) and halogen by using air, oxygen, or gas mixtures containing oxygen gas. These mixtures may include blends of oxygen with nitrogen, argon, or helium. The liberated halogen may be recycled to the halogenation reactor.

Unreacted primary mono-haloalkane may be removed from the alpha olefin or alcohol and recycled to the metathesis reactor. Recovered alpha olefin or alcohol may be further purified, as needed, to obtain the final product.

### EXAMPLE

A diol is produced by catalytic dihydroxylation of an alpha olefin with the use of hydrogen peroxide. The olefin is made according to the halogenation process described herein. Typically, 1 gram of Nafion NR50 catalyst beads (perfluorinated ion-exchange materials composed of carbon-fluorine backbone chains and perfluoro side chains containing sulfonic acid groups purchased from Aldrich Chemical Company) and 2.25 grams of 30% aqueous hydrogen peroxide (20 mmol) are mixed in a vessel such as a glass round bottom flask at room temperature and allowed to stir for 10 minutes. An olefin such as 1-octene (10 mmol) is added, the mixture is heated to 70° C. and allowed to react for 20 hours and then cooled. The mixture is filtered to separate the catalyst from the hydroxylated product. The Nafion catalyst can be washed with water, dried and reused.

1. Derivatives of alcohols and/or olefins made by halogenation of branched or n-alkanes of the same carbon number.

2. The derivatives of claim 1 wherein the branched or n-alkanes of the same carbon number are halogenated to make primary mono-haloalkanes which are then reacted with metal oxides to make the olefins and/or alcohols.

3. Derivatives of alcohols and/or olefins made by halogenation of branched or n-alkanes of the same carbon number comprising alcohol ethoxylates, alcohol sulfates, alcohol sulfated ethoxylates, surfactants, including those made with these ethoxylates and/or sulfated ethoxylates, detergents made with these surfactants, oxyalkylated alcohols, oxyalkylated alcohol sulfates, polymethacrylate esters, alkyl amines and their derivatives, linear phthalates, linear adipates, alcohol ether amines, alkyl glycerol ether sulfonates, thioproprionate esters, alkyl polyglucosides, alcohol phosphates, alcohol ether phosphates, esters of fatty acids, alcohol phosphites, benzophenones, secondary alcohol ethoxylates, secondary alcohol sulfates, secondary alcohol sulfated ethoxylates, surfactants, including those made with secondary ethoxylates and/or secondary sulfated ethoxylates, and detergents made with these surfactants, alkylbenzene, alkylxylene, alkylphenol, alkylnaphthalene, detergent alcohols, plasticizer alcohols, alkylates of phenylbenzo compounds, alkyl ammonium salts of unsaturated fatty acids, alkenyl succinates, ether secondary alcohols, diols and polyols produced by catalytic dihydroxylation of internal olefins with the use of hydrogen peroxide, internal olefins, detergent alcohols, plasticizer alcohols, alkyl amines and their derivatives, alpha olefin sulfonates, alkenyl succinates, polyalphaolefins, linear mercaptans, synthetic acids, chlorinated alpha olefins, aluminum alkyls, alkyl diphenylether disulfonates, fatty acids, lubricant additives, and ether primary alcohols.

4. The derivatives of claim 3 wherein the branched or n-alkanes of the same carbon number are halogenated to make primary mono-haloalkanes which are then oxidized with metal oxides to make the olefins and/or alcohols

5. Ethoxylated derivatives of olefins and/or alcohols, sulfated ethoxylated derivatives of olefins and/or alcohols, surfactants made with ethoxylated and/or sulfated ethoxylated derivatives of olefins and/or alcohols, detergents made with ethoxylated and/or sulfated ethoxylated derivatives of olefins and/or alcohols, and/or surfactants made with ethoxylated and/or sulfated ethoxylated derivatives of olefins and/or alcohols wherein said olefins and/or alcohols are made by a process which includes the steps of a) halogenating linear alkanes, branched alkanes, or a mixture of linear and branched alkanes to produce a mixture of mono-haloalkanes and hydrogen halide; b) separating the hydrogen halide from the mixture of step a) and optionally neutralizing it with a metal oxide to produce a partially halogenated metal oxide and/or metal halide which may be regenerated to halogen and metal oxide; c) separating the mono-haloalkanes from the mixture of step a); d) reacting the separated mono-haloalkane with a metal oxide to convert the mono-haloalkane to a mixture of products that contains olefins, alcohols, and unconverted mono-haloalkanes and a partially halogenated metal oxide and/or metal halide; and e) removing the unreacted mono-haloalkane from the reaction mixture and then purifying the olefin and/or alcohol.

6. Derivatives of alcohols and/or olefins comprising alcohol ethoxylates, alcohol sulfates, alcohol sulfated ethoxylates, surfactants, including those made with these ethoxylates and/or sulfated ethoxylates, detergents made with these surfactants, oxyalkylated alcohols, oxyalkylated alcohol sulfates, polymethacrylate esters, alkyl amines and their derivatives, linear phthalates, linear adipates, alcohol ether amines, alkyl glycerol ether sulfonates, thioproprionate esters, alkyl polyglucosides, alcohol phosphates, alcohol ether phosphates, esters of fatty acids, alcohol phosphites, benzophenones, secondary alcohol ethoxylates, secondary alcohol sulfates, secondary alcohol sulfated ethoxylates, surfactants, including those made with secondary ethoxylates and/or secondary sulfated ethoxylates, and detergents made with these surfactants, alkylbenzene, alkylxylene, alkylphenol, alkylnaphthalene, detergent alcohols, plasticizer alcohols, alkylates of phenylbenzo compounds, alkyl ammonium salts of unsaturated fatty acids, alkenyl succinates, ether secondary alcohols, diols and polyols produced by catalytic dihydroxylation of internal olefins with the use of hydrogen peroxide, internal olefins, detergent alcohols, plasticizer alcohols, alkyl amines and their derivatives, alpha olefin sulfonates, alkenyl succinates, polyalphaolefins, linear mercaptans, synthetic acids, chlorinated alpha olefins, aluminum alkyls, alkyl diphenylether disulfonates, fatty acids, lubricant additives, and ether primary alcohols wherein the olefins and/or alcohols are made by a process which includes the steps of a) halogenating linear alkanes, branched alkanes, or a mixture of linear and branched alkanes to produce a mixture of mono-haloalkanes and hydrogen halide; b) separating the hydrogen halide from the mixture of step a) and optionally neutralizing it with a metal oxide to produce a partially halogenated metal oxide and/or metal halide which may be regenerated to halogen and metal oxide; c) separating the mono-haloalkanes from the mixture of step a); d) reacting the separated mono-haloalkane with a metal oxide to convert the mono-haloalkane to a mixture of products that contains olefins, alcohols, and unconverted mono-haloalkanes and a partially halogenated metal oxide and/or metal halide; and e) removing the unreacted mono-haloalkane from the reaction mixture and then purifying the olefin and/or alcohol.
